(19) United States
(12) Patent Application Publication
(10) Pub. No.: US 2022/0265455 A1
(43) Pub. Date: Aug. 25, 2022

(54) IMPROVED MEDICAL APPARATUS FOR CONTAINING HAEMATOMAS

(71) Applicants: Ciro ANGELORO, Sant'Angelo Lodigiano (LO) (IT); Enrico MORSILLO, San Colombano al Lambro (MI) (IT)

(72) Inventors: Ciro ANGELORO, Sant'Angelo Lodigiano (LO) (IT); Enrico MORSILLO, San Colombano al Lambro (MI) (IT)

(21) Appl. No.: 17/629,776

(22) PCT Filed: Jul. 24, 2020
(86) PCT No.: PCT/IB2020/056998

§ 371 Date: Jan. 24, 2022

(30) Foreign Application Priority Data
Jul. 25, 2019 (IT) 102019000012843

(51) Int. Cl. A61F 5/058

(52) U.S. Cl. CPC A61F 5/05816; A61F 5/05858; A61N 1/372

(57) ABSTRACT

There is described a medical apparatus (1) for the containment of hematomas for treatment following operations for implanting cardiac devices. The apparatus comprises a support unit (2) externally enveloping at least a part of the patient's body and adapted to resist efforts aimed to detach said unit from said patient, at least one inflatable cushion (4) held in place over or close to the patient's shoulder by said support unit. The support unit comprises at least one pocket (10) arranged between the inflatable cushion and the patient s shoulder; the pocket comprises a material (11) configured to distribute the pressure of the cushion, once inflated, on the patient's shoulder. The inflatable cushion (40) is shaped so that, once inflated, it takes a wavy shape.

The present invention relates to a medical apparatus for improved hematoma containment.

Performing a pacemaker or defibrillator implantation in a patient exhibits post-operative risks including the formation of a hematoma in the shoulder, especially in patients who undergo therapies with the use of anticoagulants or antiplatelet agents.

The formation of a hematoma close to the pocket of the pacemaker or defibrillator can lead to the development of an infection with possible damage to the cardiac tissue.

Another complication is the displacement of the implanted leads as a result of early or accidental patient mobilization.

To combat the aforementioned complications, the patient's shoulder is currently subjected to compression bandage, which must be carried out by skilled personnel in order to be performed correctly.

A medical apparatus for the containment of hematomas comprising a support unit enveloping at least a part of the patient's body and an inflatable cushion held in place by the support unit is currently known from patent application WO 2016/001802.

However, the position of the cushion directly in contact with the patient's body is not comfortable for the patient and does not allow a uniform distribution of pressure over the whole part where the hematoma is present.

In view of the background art, it is the object of the present invention to provide a medical apparatus for the containment of hematomas which overcomes the aforementioned drawbacks.

According to the present invention, said object is achieved by a medical apparatus for the containment of hematomas for treatment following operations for implanting cardiac devices, said apparatus comprising a support unit externally enveloping at least a part of the patient's body and adapted to resist efforts aimed to detach said unit from said patient, at least one inflatable cushion held in place over or close to the patient's shoulder by said support unit, characterized in that it comprises a material arranged between said inflatable cushion and the patient's shoulder, said material being configured to distribute the pressure of said cushion, once inflated, on the patient's shoulder, said support unit comprising at least one pocket arranged to contain said material, said inflatable cushion being shaped so that, once inflated, it takes a wavy shape.

The features and advantages of the present invention will become apparent from the following detailed description of a practical embodiment thereof, shown by way of non-limiting example in the accompanying drawings, in which:

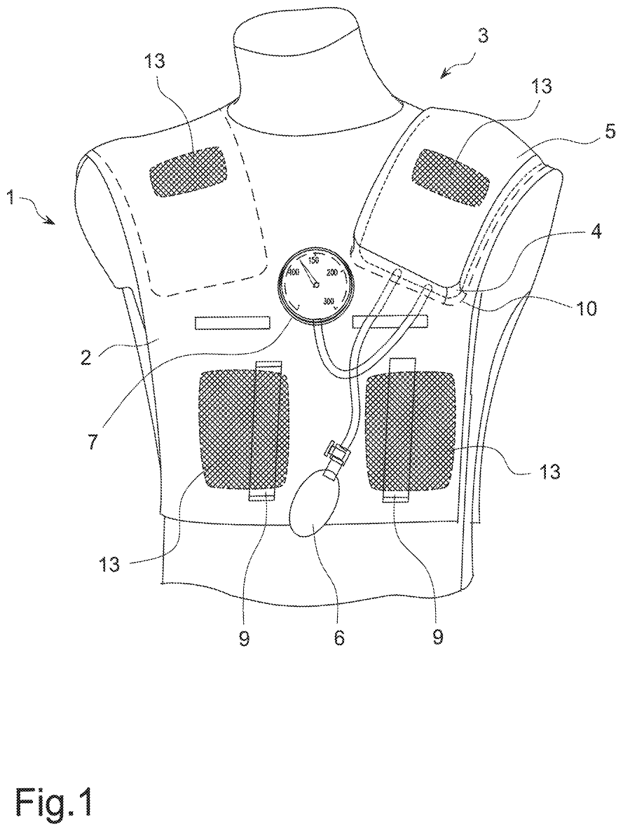

FIG. 1 is a front view of the medical apparatus for the containment of hematomas according to a first embodiment of the present invention;

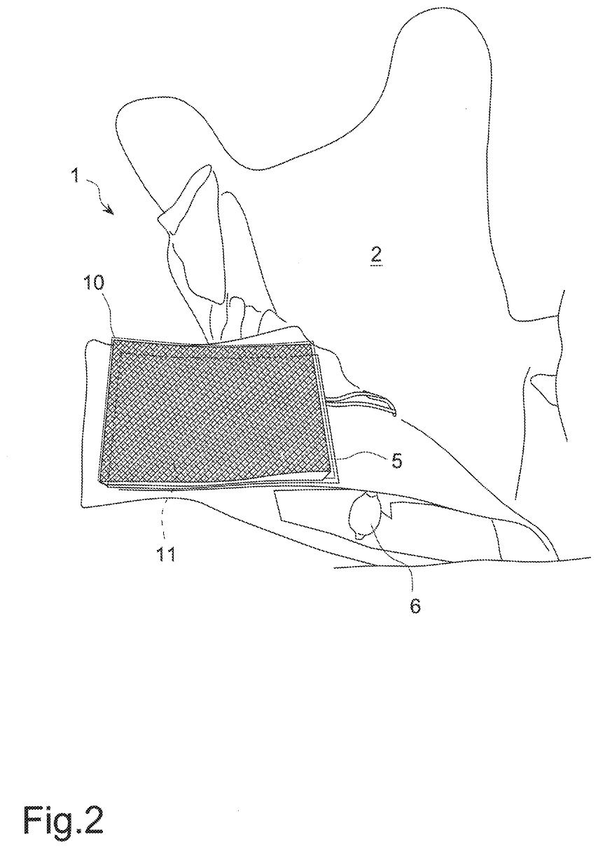

FIG. 2 is a view of the internal part of the medical apparatus in FIG. 1;

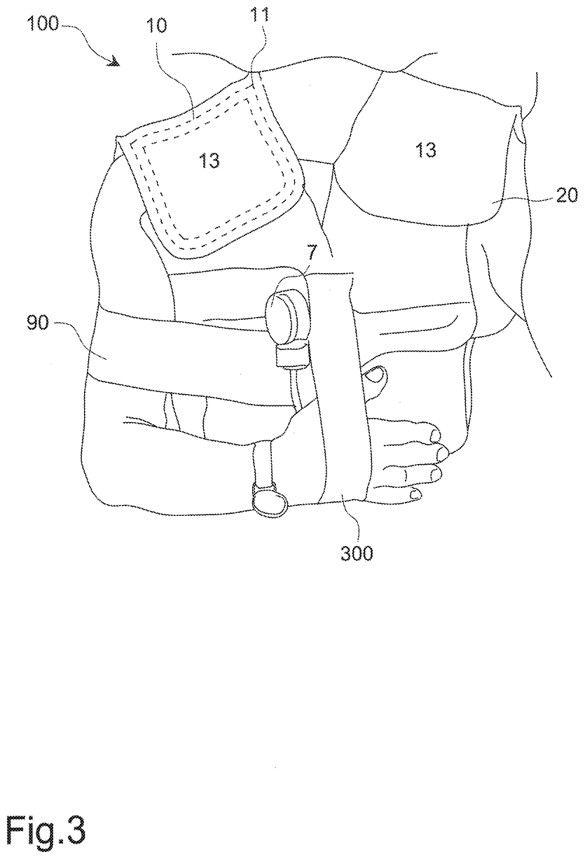

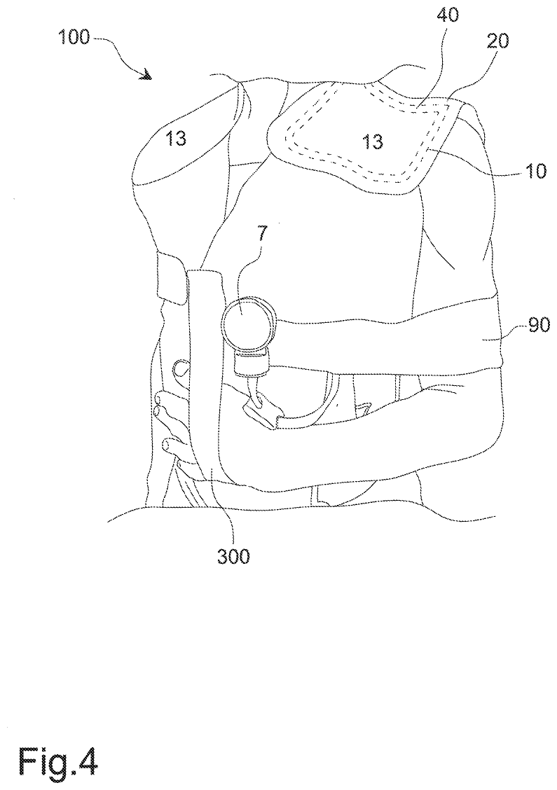

FIGS. 3 and 4 are views of the medical apparatus for the containment of hematomas according to a second embodiment of the invention worn by a patient;

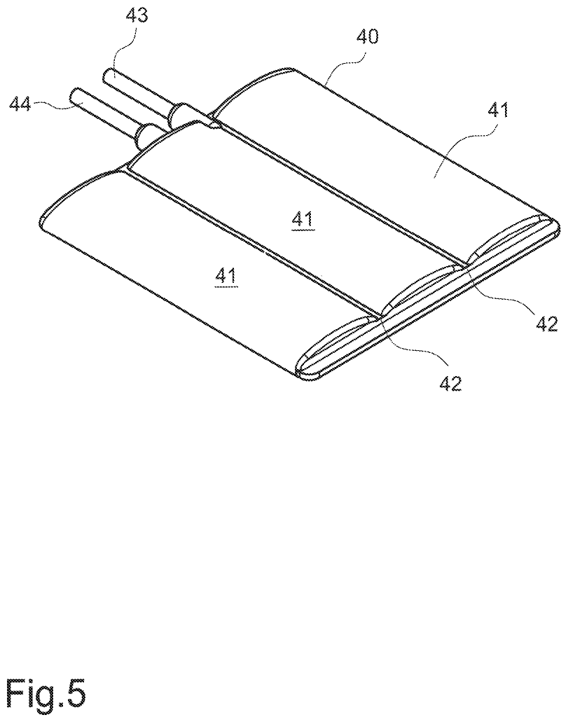

FIG. 5 is a perspective view of the inflatable cushion of the apparatus in FIGS. 3 and 4;

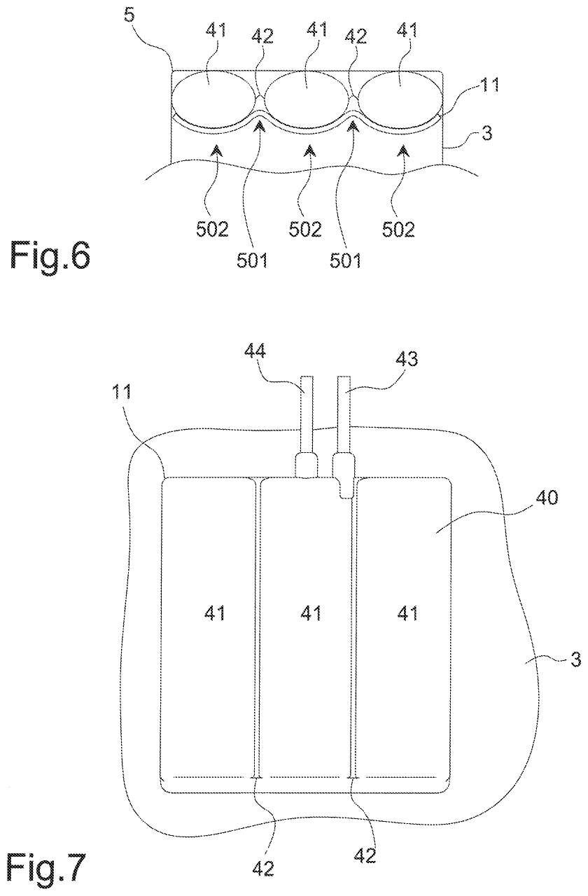

FIG. 6 is a section view of a part of the apparatus in FIGS. 3 and 4 applied to a patient with the cushion inflated;

FIG. 7 is a top view of a part of the apparatus in FIGS. 3 and 4 applied to a patient with the cushion inflated.

With reference to FIGS. 1 and 2, a medical apparatus 1 for the containment of hematomas according to a first embodiment of the present invention is described.

The apparatus comprises a support unit 2 adapted to cover and externally envelop the patient's shoulder; preferably the support unit 2 is in the form of a jacket. The support unit 2 is provided in particular for treatments following operations for implanting a pacemaker or a defibrillator, i.e., when hematomas are possible at the shoulder.

The apparatus comprises at least one bag or cushion 4 preferably inflatable with air, substantially flattened and arranged at or straddling the shoulder; the inflatable bag or cushion is held in place on the shoulder by the support unit 2.

The apparatus comprises manually activatable inflation devices 6, such as manual inflation means, and means 7 for detecting the pressure in the cushion, such as a pressure gauge.

The support unit comprises at least one pocket 5 configured to house the inflatable cushion 4. Preferably, the pressure of the inflatable cushion 4 is between 80 and 100 millimeters of mercury and the support unit 2 must bear said pressure without deforming in order to transmit said pressure to the patient's shoulder.

The support unit 2 in the form of a jacket comprises a plurality of portions 13 which are stably mutually engageable by means of connections, for example with Velcro; the portions are arranged so as to firmly grip at least one inflatable cushion 4 arranged in the pocket 5 over a shoulder.

The jacket-shaped support unit 2 comprises anchoring means 9, for example strips, adapted to support and hold a patient's arm substantially immobile.

Advantageously, the support unit 2 comprises, at the pocket 5 and in the inner part of the jacket, another pocket 10 for the containment of a material 11, for example memory foam, adapted to uniformly distribute the pressure of the inflatable cushion 4 on the shoulder 3; the pressure of the inflated cushion 4 is thus uniformly distributed over the part where the hematoma is present so as to ensure certain compression values on the entire part of the patient's body affected by the hematoma.

The support unit 2 is applied to the patient so that the same unit adheres to the patient's body, in particular in the area where the inflatable cushion 4 is placed. The latter is initially deflated and is inflated immediately after the application of the support unit 2 to the patient.

Generally, the pressure to be reached is between 80 and 100 millimeters of mercury so as to apply local compression to the patient. The material 11 allows to uniformly distribute said compression exerted by the now inflated cushion 4; moreover, the presence of the material 11 allows the patient to obtain greater comfort.

The pressure in the cushion 4 may be monitored and controlled by the pressure gauge.

With reference to FIGS. 3-7, a medical apparatus 100 for the containment of hematomas according to a second embodiment of the present invention is described. The medical apparatus 100 of the second embodiment of the invention differs from the medical apparatus 1 of the first embodiment of the invention in the different shape of the inflatable cushion 40 with respect to the inflatable cushion 4 and in the presence of some additional elements 90 and 300.

The apparatus 100 comprises a support unit 20, seen in FIGS. 3 and 4, adapted to externally cover and envelop the patient's shoulder; preferably the support unit 20 is in the form of a jacket. The support unit 20 is provided in particular for treatments following operations for implanting a pacemaker or a defibrillator, i.e., when hematomas are possible at the shoulder.

The apparatus comprises at least one bag or cushion 40 preferably inflatable with air, substantially flattened and arranged at or straddling the shoulder, as better seen in FIG. 5; the inflatable bag or cushion is held in place on the shoulder by the support unit 20. The inflatable cushion 40 is rectangular in shape and has size such as to cover the entire wound caused by the implantation of the pacemaker or defibrillator; the inflatable cushion 40 is designed so as to take a wavy shape when inflated.

In particular, the inflatable cushion 40 comprises a sequence of three sectors 41 having the same dimensions which are normally flattened but when inflated take the top hat-like or -type shape each. The sectors 41 are adjacent to one another and are joined together by long side thereof, i.e., by the cylindrical side surface when the sectors 41 are inflated, by means of junction areas 42 having the same length as the sectors 41; the inflatable cushion 40 has three sectors 41 alternated with two junction areas 42 so as to take the wavy shape once inflated. The sectors 41 are simultaneously inflatable and deflatable by a tube 43 connected to the inflation device 6 while another tube 44 is connected to the pressure detection means 7.

The particular shape of the cushion 40, once inflated, allows the pressure to be well distributed over the entire wound or hematoma of the shoulder 3, however the presence of the junction areas 42 between the sectors 41 allows the formation of areas 501 on the patient's shoulder, i.e., the areas at the junction areas 42, where the pressure exerted by the inflatable cushion is slightly lower than the areas 502 below the sectors 41, as better seen in FIGS. 6 and 7; the cushion 40 thus allows a better circulation of the blood in the areas 501 of the patient's shoulder corresponding to the junction areas 42 as compared to the areas 502 of the patient's shoulder, corresponding to the sectors 41, subjected to greater pressure. Preferably, the sectors 41 are 5 cm wide and 15.7 cm long while the junction areas are 0.25 cm wide and 15.7 cm long.

The apparatus comprises manually activatable inflation devices 6 (not seen in FIGS. 3 and 4), such as manual inflation means, and means 7 for detecting the pressure in the cushion, such as a pressure gauge.

The support unit comprises at least one pocket 5 configured to house the inflatable cushion 40. Preferably, the pressure of the inflatable cushion 40 is between 100 and 150 millimeters of mercury and the support unit 20 must bear said pressure without deforming in order to transmit said pressure to the patient's shoulder.

The support unit 20 in the form of a jacket comprises a plurality of portions 13 which are stably mutually engageable by means of connections, for example with Velcro; the portions are arranged so as to firmly grip at least one inflatable cushion 40 arranged in the pocket 5 over a shoulder. The Velcro engageable portions 13 allow a continuous monitoring of the wound of the patient who has undergone an operation for implanting a pacemaker or defibrillator.

The jacket-shaped support unit 20 comprises means adapted to support and hold a patient's arm substantially immobile. In particular, said means comprise a lateral band 90 with Velcro fastening, which band serves for the immobilization of the limb, thus reducing the complications related to the dislocation of the leads from the cardiac muscle, considering that a pacemaker or defibrillator rests on the pectoral muscle, and any unusual movement of the limb would result in the complication described above, thus making the operation of the implanted devices null.

Advantageously, the support unit 2 comprises, at the pocket 5 and in the inner part of the jacket, another pocket 10 for the containment of the material 11, for example memory foam, for a better patient comfort or ease when the cushion 40 is inflated and exerts compression on the shoulder 3.

The support unit 20 is applied to the patient so that the same unit adheres to the patient's body, in particular in the area where the inflatable cushion 40 is placed. The latter is initially deflated and is inflated immediately after the application of the support unit 20 to the patient.

Generally, the pressure to be reached is between 100 and 150 millimeters of mercury so as to apply local compression to the patient. The material 11 allows the patient to obtain greater comfort.

The pressure in the cushion 40 may be monitored and controlled by the pressure gauge 7. The support unit 20 also comprises a central support 300 for hand housing. This support has been added to prevent and counteract stasis edemas of the affected limb and hand due to the medical contraindication of limb mobility and the operation itself. This support allows to put the limb and hand in relief, preventing edema.

Furthermore, the medical apparatus 100 and components thereof and therefore the inflatable cushion 40 as well are made of radiolucent materials which allow clinical and instrumental investigations on the patient without the need to remove the apparatus itself from the patient; for example, the jacket can be made of cotton.

FIGS. 3 and 4 show that the apparatus 100 is made to adapt to whether the patient undergoes an implantation on the left or right shoulder, since the pocket 5 for the containment of the inflatable cushion 40 and the pocket 10 for the containment of the material 11 can be placed on the patient's right or left shoulder and always held in place by the support unit 20. Likewise, the band 90 can allow the immobilization of the left or right limb.

According to a variant of the first embodiment of the invention, the cushion 4 can have the same shape as the cushion 40 and be completely the same as the cushion 40; in this case, it does not perform uniform compression on the patient's shoulder where the wound or hematoma is located, but behaves as previously mentioned and shown in FIGS. 6 and 7.

1. Medical apparatus for the containment of hematomas for treatment following operations for implanting cardiac devices, said apparatus comprising a support unit externally enveloping at least a part of the patient's body and adapted to resist efforts aimed to detach said unit from said patient, at least one inflatable cushion held in place over or close to the patient's shoulder by said support unit, wherein it comprises a material arranged between said inflatable cushion and the patient's shoulder, said material being configured to distribute the pressure of said cushion, once inflated, on the patient's shoulder, said support unit comprising at least one pocket arranged to contain said material, said inflatable cushion being shaped so that, once inflated, it takes a wavy shape.

2. Medical apparatus according to claim 1, wherein said material is memory foam.

3. Medical apparatus according to claim 1, wherein said support unit is in the shape of a jacket.

4. Medical apparatus according to claim 1, wherein said inflatable cushion is arranged inside a pocket of said support unit.

5. Medical apparatus according to claim 1, wherein said medical apparatus is made of radiolucent materials.

6. Medical apparatus according to claim 1, wherein it comprises at least one lateral band for the immobilization of the limb close to said shoulder of the patient.

7. Medical apparatus according to claim 6, wherein it comprises a central support for the hand of the patient's limb.

8. Medical apparatus according to claim 1, wherein said inflatable cushion comprises a sequence of at least three sectors connected together, which, when inflated, take a cylindrical shape each, and junction areas, each between two sectors of said at least three sectors.

9. Medical apparatus according to claim 1, wherein said support unit comprises a plurality of portions which are stably mutually engageable by means of Velcro connections.
